(19) United States
(12) Patent Application Publication
(10) Pub. No.: US 2022/0162212 A1
(43) Pub. Date: May 26, 2022

(54) PROCESS FOR THE PREPARATION OF (6S)-3-[(4S)-4-CYANO-2-OXO-PYRROLIDIN-1-YL]-6-METHYL-N-(3,4,5-TRIFLUOROPHENYL)-6,7-DIHYDRO-4H-PYRAZOLO[1,5-A]PYRAZINE-5-CARBOXAMIDE

(71) Applicant: Hoffmann-La Roche Inc., Little Falls, NJ (US)

(72) Inventors: Weichun CHEN, Shanghai (CN); Lin WANG, Shanghai (CN); Qiwu ZHAO, Shanghai (CN)

(73) Assignee: Hoffmann-La Roche Inc., Little Falls, NJ (US)

(21) Appl. No.: 17/433,961

(22) PCT Filed: Feb. 25, 2020
(86) PCT No.: PCT/EP2020/054806

§ 371 Date: Aug. 25, 2021

(30) Foreign Application Priority Data
Feb. 26, 2019 (CN) PCT/CN2019/076126

(51) Int. Cl. C07D 487/04; C07D 207/277

(52) U.S. Cl. CPC C07D 487/04; C07D 207/277

(57) ABSTRACT

The present invention relates to a process for synthesizing a compound (I), or pharmaceutically acceptable salt thereof, which is useful for prophylaxis and treatment of a viral disease in a patient relating to hepatitis B infection or a disease caused by hepatitis B infection.

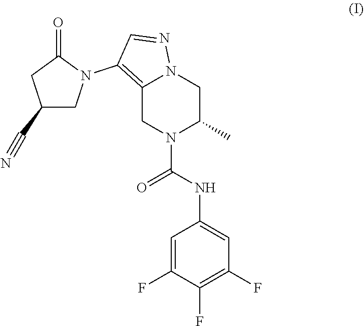 [SMILES: C[C@H]1Cn2ncc(N3C[C@@H](C#N)CC3=O)c2CN1C(=O)Nc1cc(F)c(F)c(F)c1]

The present invention relates to a process for the preparation of (6S)-3-[(4S)-4-cyano-2-oxo-pyrrolidin-1-yl]-6-methyl-N-(3,4,5-trifluorophenyl)-6,7-dihydro-4H-pyrazolo[1,5-a]pyrazine-5-carboxamide (compound (I)),

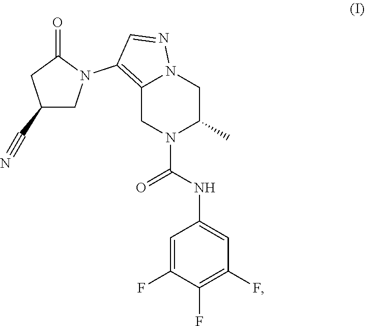 [SMILES: C[C@H]1Cn2ncc(N3C[C@@H](C#N)CC3=O)c2CN1C(=O)Nc1cc(F)c(F)c(F)c1]

or pharmaceutically acceptable salt thereof, which is useful for prophylaxis and treatment of a viral disease in a patient relating to hepatitis B infection or a disease caused by hepatitis B infection.

### BACKGROUND OF THE INVENTION

The relevant synthetic approach of compound (I) was disclosed as Example 240 in patent WO2016113273, however it is not suitable for commercial process due to the following issues:
1. chiral separation was required;
2. excessive and toxic I₂ was not removed during the synthesis of tert-butyl (6S)-3-iodo-6-methyl-6,7-dihydro-4H-pyrazolo[1,5-a]pyrazine-5-carboxylate, which makes it highly dangerous for large scale manufacture;
3. if chiral starting material ((3S)-5-oxopyrrolidine-3-carbonitrile) was used, the chirality is susceptible to the Ullmann reaction condition and intent to undergo epimerization.

Based on the issues above, one object of the invention therefore is to find an alternative efficient synthetic approach which can be applied on a technical scale and/or result in obtaining the product in a higher yield and/or desired purity as well as required chiral configuration. Addressing any of the issues mentioned above is also one of the objects of the invention.

Furthermore, the advantage of current process of this invention over the previous one disclosed in the prior art are:
1. no chiral separation is needed, and the synthesis of chiral starting material ((3S)-5-oxopyrrolidine-3-carbonitrile) is firstly reported in current invention, which can be easily made in large scale and therefore shortens the whole process;
2. excessive and toxic 1₂ was removed in step 1) with reducing agent compared to prior art, the amount of which was then carfully controlled to avoid loss of product;
3. Ullmann reaction condition was carefully optimized to keep the chirality of (3S)-5-oxopyrrolidine-3-carbonitrile moiety unchanged throughout the reaction;
4. the de-BOC reaction was performed in an environment friendly condition.

### DETAILED DESCRIPTION OF THE INVENTION

### Definitions

The term “pharmaceutically acceptable salt” refers to conventional acid-addition salts or base-addition salts that retain the biological effectiveness and properties of the compounds of formula I and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described in Bastin R. J., et al., Organic Process Research & Development 2000, 4, 427-435; or in Ansel, H., et al., In: Pharmaceutical Dosage Forms and Drug Delivery Systems, 6th ed. (1995), pp. 196 and 1456-1457.

### Abbreviation

ACN Acetonitrile
eq Equivalent
DBU 1,8-Diazabicyclo[5.4.0]undec-7-ene
DCM Dichloromethane
DIPEA N,N-Diisopropylethylamine
DMEDA N,N′-Dimethyl-1,2-ethanediamine
IPA Isopropyl alcohol
IPAc Isopropyl acetate
MeTHF 2-Methyl tetrahydrofuran
MTBE Methyl tert-butyl ether
NIS N-Iodosuccinimide
TEA Triethylamine
TFA Trifluoroacetic acid
TFAA Trifluoroacetic anhydride
V volume
wt % weight percent

The present invention provides the process for preparing the compound (I) as outlined in the Scheme 1 and compound (IV) as outlined in the Scheme 2.

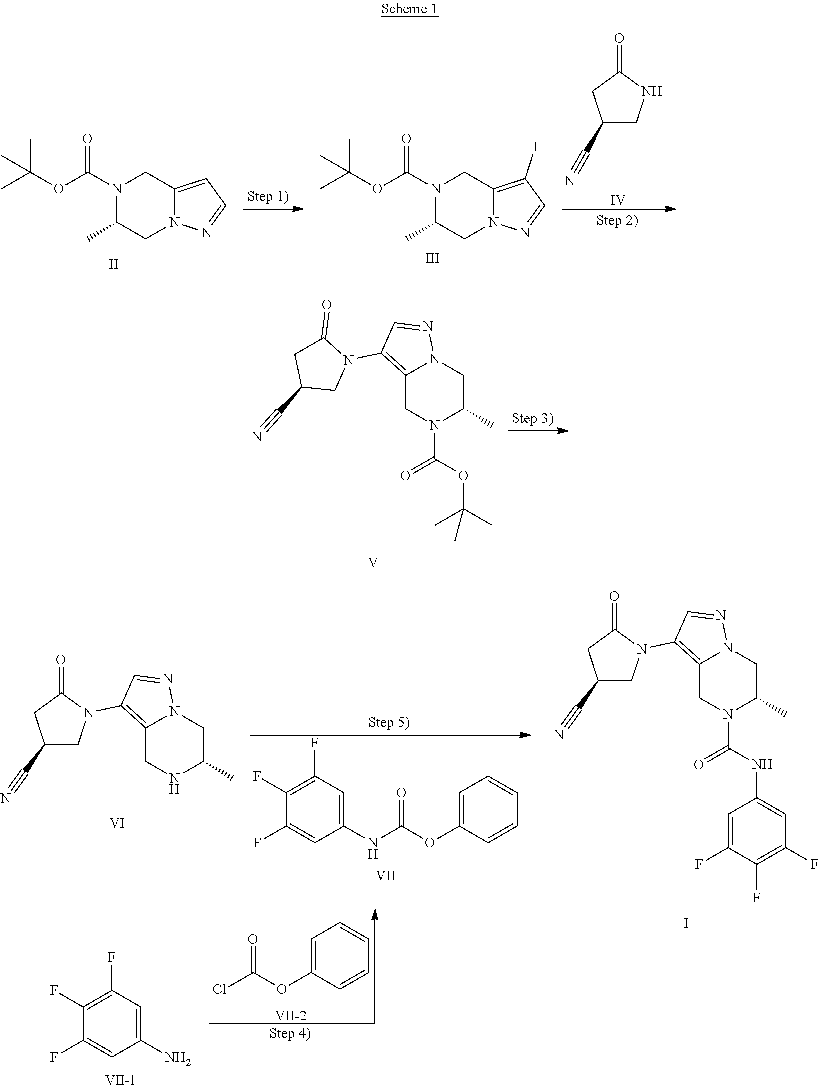

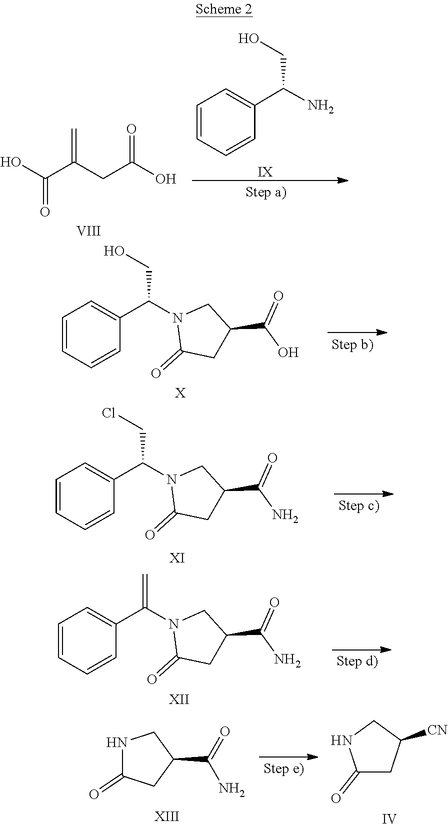

The synthesis of compound (I) comprises one or more of the following steps:

step 1) the formation of compound (III),

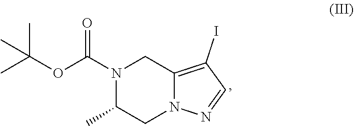 [SMILES: C[C@H]1Cn2ncc(I)c2CN1C(=O)OC(C)(C)C]

via iodization reaction of compound (II),

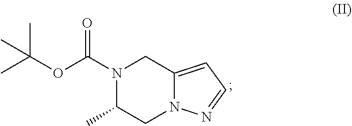 [SMILES: C[C@H]1Cn2nccc2CN1C(=O)OC(C)(C)C]

step 2) the formation of compound (V),

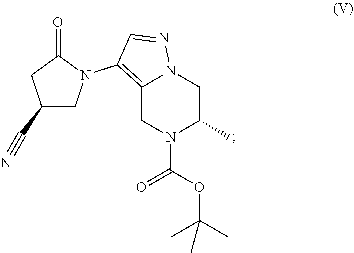 [SMILES: C[C@H]1Cn2ncc(N3C[C@@H](C#N)CC3=O)c2CN1C(=O)OC(C)(C)C]

via Ullmann reaction between compound (III) and compound (IV),

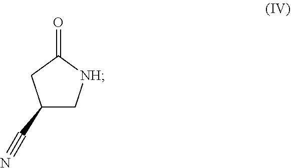

step 3) the formation of compound (VI),

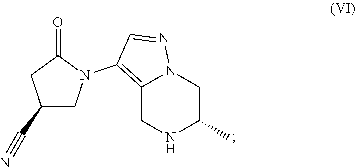 [SMILES: C[C@H]1Cn2ncc(N3C[C@@H](C#N)CC3=O)c2CN1]

via de-protection of compound (V);

step 4) the formation of compound (VII),

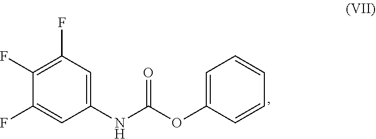 [SMILES: O=C(Nc1cc(F)c(F)c(F)c1)Oc1ccccc1]

via the reaction between 3,4,5-trifluoroaniline and phenyl carbonochloridate;

step 5) the formation of compound (I),

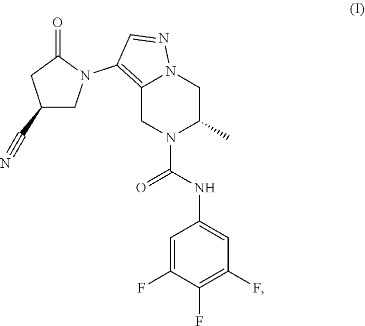 [SMILES: C[C@H]1Cn2ncc(N3C[C@@H](C#N)CC3=O)c2CN1C(=O)Nc1cc(F)c(F)c(F)c1]

via the substitution reaction between compound (VI) and compound (VII).

A detailed description of synthesis of compound (I) in present invention of process steps is as following:

Step 1) the formation of compound (III),

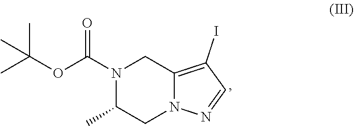 [SMILES: C[C@H]1Cn2ncc(I)c2CN1C(=O)OC(C)(C)C]

via iodization reaction of compound (II),

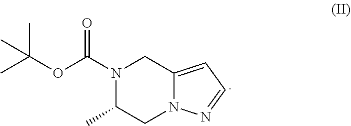 [SMILES: C[C@H]1Cn2nccc2CN1C(=O)OC(C)(C)C]

The formation of compound (III) is usually performed in the presence of a suitable iodization reagent and a suitable organic solvent. The conversion as a rule is performed under room temperature or heating condition.

The suitable iodization reagent is selected from NIS and I₂, particularly the reagent is NIS. The excessive I₂ formed during the reaction due to the use of iodization reagent was removed by addition of Na₂SO₃ with amount of 0.4˜0.8 eq., particularly 0.55˜0.6 eq.

The suitable organic solvent is selected from DCM, THF, ACN and MeTHF, particularly the organic solvent is ACN.

The iodization reaction as a rule is performed at 10˜60° C., particularly at 25˜30° C.

The reaction was quenched by Na₂SO₃ to remove excessive I₂ formed during the reaction, which is very important for the safety of large scale manufacture. However, it was found during the test that excessive Na₂SO₃ will reduce the product and convert compound (III) back to compound (II), while inadequate Na₂SO₃ can not efficiently quench the reaction. A series of tests were done and summarized in the table below, it was concluded that 0.55˜0.6 eq. of Na₂SO₃ can meet the above criteria and therefore considered as the optimal condition.

TABLE 1
tests on the impact of the amount of Na₂SO₃

| Test No. | Eq. of Na₂SO₃ | Results |
|---|---|---|
| 1 | 0.4 | The reaction was not |
| | | quenched sufficiently, the |
| | | color of the product was dark |
| 2 | 0.55 | 0.2% reduction impurity was |
| | | found once workup was done |
| 3 | 0.6 | 0.5% reduction impurity was |
| | | found once workup was done |
| 4 | 0.8 | 2% reduction impurity was |
| | | found once workup was done |

Step 2) the formation of compound (V),

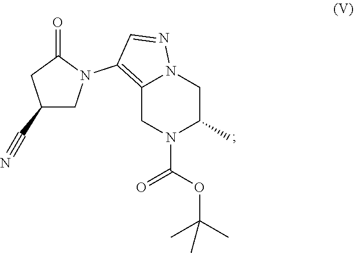 [SMILES: C[C@H]1Cn2ncc(N3C[C@@H](C#N)CC3=O)c2CN1C(=O)OC(C)(C)C]

via Ullmann reaction between compound (III) and compound (IV),

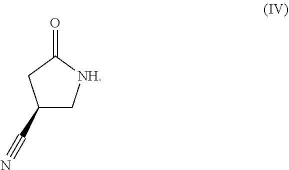

The formation of compound (V) is usually performed in the presence of a suitable base, a suitable catalyst, a suitable ligand and a suitable organic solvent. The conversion as a rule is performed under a heating condition.

The suitable base is selected from K₂CO₃, K₃PO₄ and Cs₂CO₃, particularly the base is K₂CO₃.

The suitable catalyst is CuI and the amount of catalyst is 0.05-0.5eq, particularly 0.1 eq.; the suitable ligand is DMEDA and the amount of ligand is 0.2-2.0 eq., particularly 1.0 eq.

The suitable organic solvent is selected from 1,4-Dioxane, ACN, Toluene, THF and MeTHF, particularly the organic solvent is THF.

The dehydration reaction as a rule is performed at 60° C.˜120° C., particularly at 70° C.˜75° C.

The chiral center next to cyano group of compound (V) is intent to epimerize under the Ullmann reaction condition, therefore it is necessary to optimize the condition to keep the chirality of compound (V) steady during the reaction. However, it is very complicated to manage reaction parameters, such as base, solvent, catalyst and ligand loading, reaction time, to achieve the best conversion as well as the chiral purity. The following tests were performed to solve the above problem.

i) The impact of bases on the conversion and chiral

TABLE 2
Test of the bases

| | | Check point | | |
|---|---|---|---|---|
| Test | | (reaction | Con- | Chiral |
| No. | Reaction conditions | time) | version | purity |
| 1 | 0.5 g compound (III)/1.5 eq. | 4 h | 50% | 97.2% |
| | compound (IV)/0.1eq. CuI/0.2 | 6 h | 50% | 97.6% |
| | eq. DMEDA/2eq. | 21 h | 74% | 95.7% |
| | K₃PO₄/MeCN (10V)/50~55° C. | 28 h | 77% | 93.4% |
| | | 46 h | 88% | 92.0% |
| 2 | 1) 0.5 g compound (III)/1.5 eq. | 5 h | 43.7% | 97.5% |
| | compound (IV)/0.1eq CuI/0.2 | 26 h | 44.2% | 97.5% |
| | eq DMEDA/2eq. K₂CO₃/ | | | |
| | MeCN (10V)/ 50~55° C. | | | |
| | 2) another batch of 0.1 eq | 28.5 h | 68.7% | — |
| | CuI/0.2 eq DMEDA were | 46.5 h | 70% | — |
| | added | | | |
| | 3) another batch of 0.2 eq | 51.5 h | >99% | 97.8% |
| | CuI/0.4 eq DMEDA were | 73.5 h | >99% | 97.5% |
| | added | | | |
| 3 | 0.5 g compound (III)/1.5 eq. | 5 h | 4% | — |
| | compound (IV)/0.1 eq. CuI/0.2 | 21 h | 8% | 50% |
| | eq. DMEDA/2eq. Cs₂CO₃/ | | | |
| | MeCN (10V)/50~55° C. | | | |

Based on above test, Cs₂CO₃ can not achieve enough conversion; K₃PO₄ may achieve higher conversion but chiral purity dropped in the meantime; although K₂CO₃ did not achieve satisfactory conversion at beginning, but the conversion can be boosted by adding more catalyst and ligand, and chiral purity was stable and excellent throughout the whole test. Therefore, K₂CO₃ was selected as the suitable base.

ii) The impact of solvents on the conversion and chiral purity

TABLE 3
Test of the solvents

| | | Check point | | |
|---|---|---|---|---|
| Test | | (reaction | Con- | Chiral |
| No. | Reaction conditions | time) | version | purity |
| 1 | 0.5 g compound (III)/1.5 eq. | 18 h | 20% | 99% |
| | compound (IV)/0.1 eq. CuI/0.2 | | | |
| | eq. DMEDA/2eq. K₂CO₃/ | | | |
| | MeTHF (10V)/ 50~55° C. | | | |
| 2 | 1) 0.5g compound (III)/ 1.5 eq. | 18 h | 30% | 98% |
| | compound (IV)/0.1eq CuI/0.2 | | | |
| | eq DMEDA/ 2 eq. K₂CO₃/ | | | |
| | THF (10V)/50~55° C. | | | |
| | 2) another batch of 0.2 eq | 25 h | 89.6% | 98.1% |
| | CuI/0.4 eq DMEDA were | | | |
| | added | | | |
| | 3) another batch of 0.1 eq | 43 h | 97.5% | 97.5% |
| | CuI/0.2 eq DMEDA were | | | |
| | added | | | |
| 3 | 0.5 g compound (III)/1.5 eq. | 0 | — | — |
| | compound (IV)/0.1 eq. CuI/0.2 | | | |
| | eq. DMEDA/2eq. | | | |
| | K₂CO₃/Toluene | | | |
| | (10V)/50~55° C. | | | |

Based on above tests, the test with THF (Table 3, Test No. 2, item 1)) has higher conversion than the test with MeTHF (Table 3, Test No. 1) at 18 h. By comparing the tests with THF (Table 3, Test No. 2) and MeCN (Table 2, Test No. 2), when the final amount of CuI and DMEDA were reached same, the test with THF took shorter reaction time to achieve similar conversion than the test with MeCN. Therefore, THF was selected as the suitable solvent.

iii) The impact of catalyst and ligand loading on the conversion and chiral purity

TABLE 4
Test of catalyst and ligand loading

| | | Check point | | |
|---|---|---|---|---|
| Test | | (reaction | Con- | Chiral |
| No. | Reaction conditions | time) | version | purity |
| 1 | 1) 50g/0.1 eq CuI*/0.2 eq | l h | 42.2% | |
| | DMEDA, 3 eq. K₂CO₃ | | | |
| | powder, THF (8V), 68° C., | | | |
| | 2) another batch of 1.8 eq | 3 h | 54.4% | 99.4% |
| | DMEDA dissolved in THF | 18 h | 91.3% | |
| | (2V) was added dropwise over | | | |
| | 2 h at 68° C., then refluxing | | | |
| 2 | 1) 50 g/0.1eq CuI*/1 eq | l h | 27% | |
| | DMEDA, 3 eq. K₂CO₃ | | | |
| | powder, THF (8V), 68° C. | | | |
| | 2) another batch of 1 eq | 3 h | 33% | 98.8% |
| | DMEDA dissolved in THF | 18 h | 88.2% | |
| | (2V) was added dropwise over | | | |
| | 2 h at 68° C., then refluxing | | | |
| 3 | 1) 50g/ 0.1 eq CuI*/0.2 eq | l h | 35.3% | |
| | DMEDA, 3 eq. K₂CO₃ | | | |
| | powder, THF (8V), 68° C. | | | |
| | 2) another batch of 0.8 eq | 3 h | 50.4% | 98.8% |
| | DMEDA dissolved in THF | 20 h | 85% | |
| | (2V) was added dropwise over | | | |
| | 2 h at 68° C., then refluxing | | | |
| 4 | 1) 5g/ 0.1 eq CuI*/0.2 eq | 3 h | 69% | |
| | DMEDA, 3 eq. K₂CO₃ | | | |
| | powder, THF (10V), 68° C., | | | |
| | 2) 0.3 eq DMEDA added | 5 h | 80% | 99.7% |
| | 3) 0.3 eq DMEDA added | 18 h | 87.3% | |
| 5 | 1) 50g/0.1 eq CuI*/0.2 eq | l h | 64.5% | |
| | DMEDA, 3 eq. K₂CO₃ | | | |
| | powder, THF (8V), 68° C. | | | |
| | 2) 0.8 eq DMEDA dissolved in | 3 h | 80% | 99.5% |
| | THF (2V) was added dropwise | 20 h | 91.7% | |
| | over 2 h at 68° C., then | | | |
| | refluxing | | | |

*CuI in this test was obtained from Sundia, which has much higher activity than those previously used.

Based on the test No. 1-3 above, the ligand loading does not impact on the conversion and chiral purity too much, and it is finally determined as 1.0 eq. as the optimal ligand loading due to cost reason. By comparing the test of No. 2 in Table 1, No. 2 in Table 3, No. 4 and 5 in Table 4, it is clear that increasing the catalyst loading could increase the conversion and shorten the reaction time, however 0.1 eq. of catalyst loading is already enough to reach satisfactory conversion and chiral purity. Meanwhile, minimum catalyst loading could be environmental friendly and beneficial during heavy metal removal.

Step 3) the formation of compound (VI),

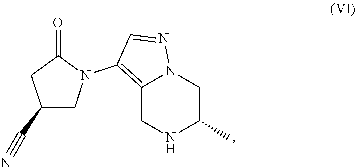 [SMILES: C[C@H]1Cn2ncc(N3C[C@@H](C#N)CC3=O)c2CN1]

via de-protection of compound (V).

The formation of compound (VI) is usually performed in the presence of a suitable acid and a suitable solvent. The conversion as a rule is performed under room temperature or a heating condition.

The suitable acid is selected from H₃PO₄, NH₄Cl, TFA and acetic acid, particularly the acid is acetic acid and the amount of acid is 1˜3 eq., particularly 1.5˜2 eq.

The suitable solvent is selected from DCM, EtOH, water and toluene, particularly the solvent is water.

The de-protection reaction as a rule is performed at room temperature or 60˜100° C., particularly at 90˜95° C.

Step 4) the formation of compound (VII),

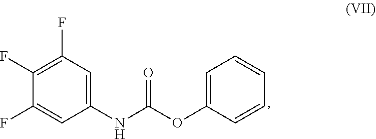 [SMILES: O=C(Nc1cc(F)c(F)c(F)c1)Oc1ccccc1]

via the reaction between 3,4,5-trifluoroaniline and phenyl carbonochloridate.

The formation of compound (VII) is usually performed in the presence of a suitable base and a suitable organic solvent. The conversion as a rule is performed under a cooling condition.

The suitable base is selected from K₂CO₃, KHCO₃, NaHCO₃ and Na₂CO₃, particularly the base is NaHCO₃.

The suitable organic solvent is selected from MTBE, IPAc, Dioxane, MeTHF and THF, particularly the organic solvent is THF.

The reaction as a rule is performed at −10˜10° C., particularly at ˜5˜0° C.

Step 5) the formation of compound (I),

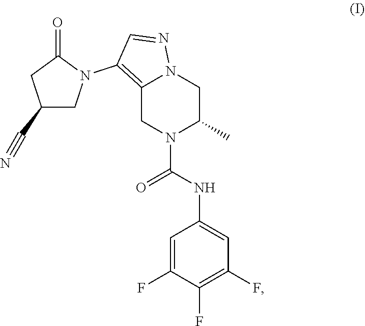 [SMILES: C[C@H]1Cn2ncc(N3C[C@@H](C#N)CC3=O)c2CN1C(=O)Nc1cc(F)c(F)c(F)c1]

via the substitution reaction between compound (VI) and compound (VII).

The formation of compound (I) is usually performed in the presence of a suitable base and a suitable organic solvent. The conversion as a rule is performed under a heating condition.

The suitable base is selected from K₂CO₃, K₃PO₄, DIPEA and TEA, particularly the base is DIPEA.

The suitable organic solvent is selected from MTBE, EA, IPAc, MeTHF and a mixture of IPAc/acetone, particularly the organic solvent is a mixture of IPAc/acetone.

The substitution reaction as a rule is performed at 30˜80° C., particularly at 45˜50° C.

The synthesis of compound (IV) comprises one or more of the following steps:

step a) the formation of compound (X),

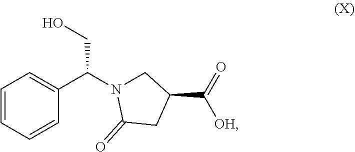 [SMILES: O=C(O)[C@H]1CC(=O)N([C@@H](CO)c2ccccc2)C1]

via cyclization reaction between compound (VIII),

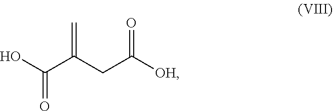

and compound (IX),

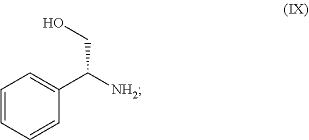

step b) the formation of compound (XI),

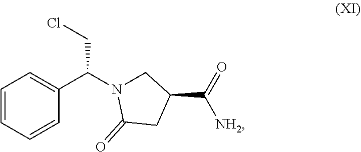 [SMILES: NC(=O)[C@H]1CC(=O)N([C@@H](CCl)c2ccccc2)C1]

via substitution of compound (X);

step c) the formation of compound (XII),

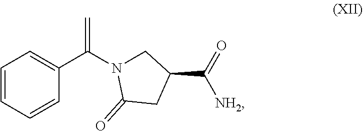 [SMILES: C=C(c1ccccc1)N1C[C@@H](C(N)=O)CC1=O]

via elimination reaction of compound (XI);

step d) the formation of compound (XIII),

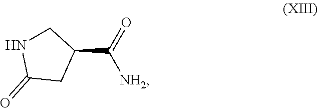

by deprotection of compound (XII);

step e) the formation of compound (IV),

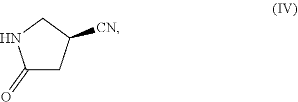

via dehydration of compound (XIII).

A detailed description of synthesis of compound (IV) in present invention of process steps is as following:

Step a) the formation of compound (X),

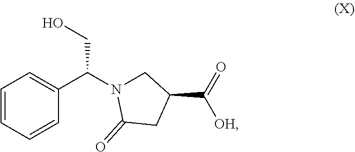 [SMILES: O=C(O)[C@H]1CC(=O)N([C@@H](CO)c2ccccc2)C1]

via cyclization reaction between compound (VIII),

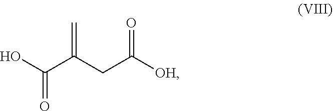

and compound (IX),

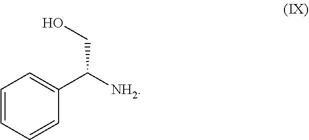

The formation of compound (X) is usually performed in the presence of a suitable organic solvent. The conversion as a rule is performed under a heating condition.

The suitable organic solvent is selected from THF, ACN and MeTHF, particularly the organic solvent is ACN.

The cyclization reaction as a rule is performed at 80˜140° C., particularly at 100˜110° C.

Step b) the formation of compound (XI),

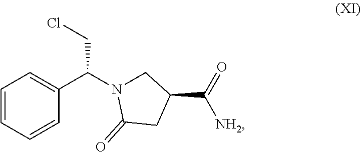 [SMILES: NC(=O)[C@H]1CC(=O)N([C@@H](CCl)c2ccccc2)C1]

via substitution of compound (X).

The formation of compound (XI) is usually performed in the presence of a chlorination reagent and a suitable organic solvent. The conversion as a rule is performed under room temperature.

The suitable chlorination reagent is selected from acetyl chloride, thionyl chloride and oxalyl chloride, particularly the reagent is thionyl chloride, and the amount of the chlorination reagent is 1˜5 eq., particularly 3 eq.

The suitable organic solvent is selected from IPAc, DCM, Toluene, THF and MeTHF, particularly the organic solvent is DCM.

The substitution reaction as a rule is performed at 10˜40° C., particularly at 20˜25° C.

Step c) the formation of compound (XII),

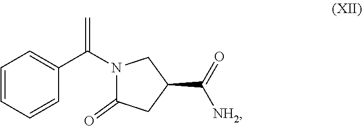 [SMILES: C=C(c1ccccc1)N1C[C@@H](C(N)=O)CC1=O]

via elimination reaction of compound (XI);

The formation of compound (XII) is usually performed in the presence of a suitable base and a suitable solvent. The conversion as a rule is performed under a heating condition.

The suitable base is selected from K₃PO₄, TEA, DBU and DIPEA, particularly the base is DBU, and the amount of base is 1˜2 eq., particularly 1.5 eq.

The suitable solvent is selected from DCM, ACN, THF and Me-THF, particularly the solvent is THF.

The elimination reaction as a rule is performed at 20˜80° C., particularly at 40˜45° C.

Step d) the formation of compound (XIII),

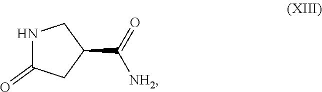

by deprotection of compound (XII);

The formation of compound (XIII) is usually performed in the presence of a suitable acid and a suitable organic solvent. The conversion as a rule is performed under room temperature.

The suitable acid is selected from H₂SO₄, H₃PO₄, HCl and TFA, particularly the acid is HCl, and the amount of acid is 0.05˜0.5 eq., particularly 0.1 eq.

The suitable organic solvent is selected from EtOH, MeOH, IPA and IPAc, particularly the organic solvent is MeOH.

The deprotection reaction as a rule is performed under room temperature, particularly at 20˜25° C.

Step e) the formation of compound (IV),

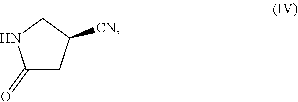

via dehydration of compound (XIII).

The formation of compound (IV) is usually performed in the presence of a suitable dehydration reagent and a suitable organic solvent. The conversion as a rule is performed under room temperature.

The suitable dehydration reagent is selected from P₂O₅, TFAA and acetic anhydride, particularly the dehydration reagent is TFAA, and the amount of dehydration reagent is 2.5 eq.

The suitable organic solvent is selected from EA, IPAc, MTBE and MeTHF, particularly the organic solvent is MTBE.

The dehydration reaction as a rule is performed under room temperature, particularly at 20˜25° C.

### EXAMPLES

### Example 1

### tert-butyl (6S)-3-iodo-6-methyl-6,7-dihydro-4H-pyrazolo[1,5-a]pyrazine-5-carboxylate (Compound (III))

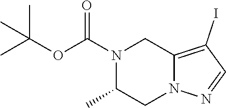 [SMILES: C[C@H]1Cn2ncc(I)c2CN1C(=O)OC(C)(C)C]

To a 1000 L jacket reactor was charged with tert-butyl (6S)-6-methyl-6,7-dihydro-4H-pyrazolo[1,5-a]pyrazine-5-carboxylate (14.1 kg, 59.5 mol, WuXi AppTec) and ACN (115 kg) at room temperature. To the mixture was then charged with NIS (20.0 kg, 88.9mol) at room temperature under N₂ protection. After being stirred at 20˜30° C. for 18 hours, the reaction mixture was slowly cooled to −5˜5° C., and aqueous Na₂SO₃ solution (1.5 wt %, 4.3 kg) was added to quench the reaction. Then to the mixture was charged with water (140 kg) slowly over 1 hour at 5˜25° C., the resulting mixture was stirred at 5˜25° C. for 1 h. The resulting suspension was separated via centrifuge in portions to collect wet cake. The wet cake was washed with ACN/water (22.3 kg, v/v=1/2.4) again, then dried using air oven (45˜50° C.) for 48 hours to give the desired product as a white solid, which was directly used for the preparation of compound (V), giving 19.4 kg of tert-butyl (6S)-3-iodo-6-methyl-6,7-dihydro-4H-pyrazolo[1,5-a]pyrazine-5-carboxylate. The purity was 99.7%, the yield was 90%. MS m/e=363.2 [M+H]⁺. ¹H-NMR (400 MHz, DMSO-d⁶) δ=7.78; (s, 1H), 4.73; (m, 1H), 4.54; (d, J=16.8 Hz, 1H), 4.42; (dd, J=12.1, 12.5 Hz, 1H), 4.07; (dd, J=6.8, 12.1 Hz, 1H), 3.69; (d, J=16.8 Hz, 1H), 1.53; (d, J=6.6 Hz, 3H), 1.41; (s, 9H).

### Example 2

### (3S)-5-oxopyrrolidine-3-carbonitrile (Compound (IV))

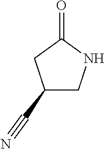

### Step a) Synthesis of (3S)-5-oxo-1-[(1R)-2-hydroxy-1-phenyl-ethyl]pyrrolidine-3-carboxylic acid (Compound (X))

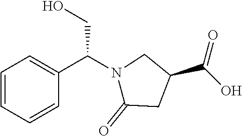 [SMILES: O=C(O)[C@H]1CC(=O)N([C@@H](CO)c2ccccc2)C1]

To a 1000 L jacket reactor was charged with Itaconic acid (62.5 kg, 480 mol, Qingdao Kehai Co. LTD), (R)-(-)-2-Phenylglycinol (70 kg, 510 mol, Jiangsu Senxuan pharmaceutical chemical co., LTD) and ACN (450 kg) at room temperature under N₂ atmosphere. The mixture was heated to 100˜110° C. and the agitation was maintained for 5 hours. Then the mixture was cooled to 5˜10° C. and the agitation was maintained for 3 hours. The solid was filtered and washed with ACN (25 kg) to give the desired product as light yellow solid, which was directly used for the preparation of Compound XI, giving 31 kg of (3S)-5-oxo-1-[(1R)-2-hydroxy-1-phenyl-ethyl]pyrrolidine-3-carboxylic acid. The purity was 98.7%, the chiral purity was 99.7%, the yield was 25%, and the MS m/e=250.1 [M+H]⁺.¹H-NMR (400 MHz, DMSO-d⁶) δ 7.40; (d, 1H), 7.40; (d, 1H), 7.38; (dd, 1H), 7.38; (dd, 1H), 7.27; (t, 1H), 5.83; (dd, 1H), 4.05; (m, 1H), 3.98; (m, 1H), 3.58; (dd, 1H, J=8.5, 13.8 Hz), 3.39; (dd, 1H, J=8.5, 13.8 Hz), 3.15; (m, 1H), 2.71; (dd, 1H, J=9.4, 18.8 Hz), 2.51; (dd, 1H, J=9.3, 18.8 Hz).

### Step b) Synthesis of (3S)-5-oxo-1-[(1R)-2-chloro-1-phenyl-ethyl]pyrrolidine-3-carboxamide (Compound (XI))

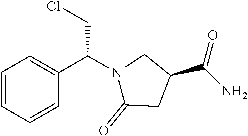 [SMILES: NC(=O)[C@H]1CC(=O)N([C@@H](CCl)c2ccccc2)C1]

To a 500 L jacket reactor was charged with (3S)-5-oxo-1-[(1R)-2-hydroxy-1-phenyl-ethyl]pyrrolidine-3-carboxylic acid (23.2 kg, 93.2 mol) and DCM (162.4 kg) at room temperature under N₂ atmosphere. To the stirred mixture was slowly charged with thionyl chloride (31 kg, 260.5 mol) at room temperature. The mixture was agitated at 20˜25° C. for another 4 hours. The reaction mixture was concentrated in vacuo at 45° C. (0.09-0.1 MPa) until no solvent is distilled out. To the residue was charged with DCM (162.4 kg) and ammonia (7 kg, 410 mol) at below 20° C. The mixture was stirred at 20˜25° C. for 16 hours. To the mixture was charged with water (115 kg) and HOAc (3.5 kg) to adjust pH to 6-7. Then the mixture was concentrated under vacuo at 30˜40° C. to evaporate DCM. Then the mixture was cooled to 10˜15° C. and stirred at 10˜15° C. for 2 hours. The solid was filtered and washed with water (50 kg) to give the desired product as light yellow solid, which was directly used for the preparation of Compound XII, giving 22 kg of (3S)-5-oxo-1-[(1R)-2-chloro-1-phenyl-ethyl]pyrrolidine-3-carboxamide. The purity was 98%, the chiral purity was 99.9%, the yield was 88%, and the MS m/e=267.1 [M+H]⁺. ¹H-NMR (400 MHz, DMSO-d⁶) δ 7.38; (dd, 1H, J=7.3, 7.4 Hz), 7.38; (dd, 1H, J=7.3, 7.4 Hz), 7.34; (d, 1H, J=7.4 Hz), 7.34; (d, 1H, J=7.4 Hz), 7.25; (t, 1H, J=7.3 Hz), 5.63; (dd, 1H, J=3.3, 8.3 Hz), 3.88; (dd, 1H, J=3.3, 12.4 Hz), 3.69; (dd, 1H, J=8.3, 12.4 Hz), 3.33; (dd, 1H, J=8.5, 13.8 Hz), 3.29; (dd, 1H, J=8.5, 13.8 Hz), 3.21; (m, 1H), 2.59; (dd, 1H, J=9.4, 18.8 Hz), 2.21; (dd, 1H, J=9.3, 18.8 Hz).

### Step c) Synthesis of (3S)-5-oxo-1-(1-phenylvinyl)pyrrolidine-3-carboxamide (Compound (XII))

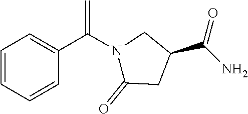 [SMILES: C=C(c1ccccc1)N1C[C@@H](C(N)=O)CC1=O]

To a 200 L jacket reactor was charged with (3S)-5-oxo-1-[(1R)-2-chloro-1-phenyl-ethyl]pyrrolidine-3-carboxamide (22 kg, 82.5 mol), THF (88 kg) and DBU (18.5 kg, 121.6 mol) at room temperature under N₂ atmosphere. The mixture was agitated using a magnetic stirrer at 40˜45° C. for 8 hours. Then the mixture was charged with HOAc (2.7 kg, 45.4 mol) to adjust pH to 6-7. The mixture was concentrated under vacuum at 40˜45° C. to evaporate excess of THF. To the mixture was charged with water (40 kg) and the mixture was agitated at 10˜15° C. for 3 hours. The solid was filtered and washed with water (25 kg) to give the desired product as light yellow solid, which was directly used for the preparation of Compound XIII, giving 15.4 kg of (3S)-5-oxo-1-(1-phenylvinyl)pyrrolidine-3-carboxamide. The purity was 99%, the chiral purity was 99.9%, the yield was 82%, and the MS m/e=231.1 [M+H]⁺. ¹H-NMR (400 MHz, DMSO-d⁶) δ 7.39; (t, 1H, J=7.3 Hz), 7.33; (dd, 1H, J=7.3, 7.4 Hz), 7.33; (dd, 1H, J=7.3, 7.4 Hz), 7.28; (d, 1H, J=7.4 Hz), 7.28; (d, 1H, J=7.4 Hz), 4.77; (s, 1H), 4.57; (s, 1H), 3.77; (dd, 1H, J=8.5, 13.8 Hz), 3.48; (dd, 1H, J=8.5, 13.8 Hz), 3.20; (m, 1H), 2.59; (dd, 1H, J=9.4, 18.8 Hz), 2.21; (dd, 1H, J=9.3, 18.8 Hz).

### Step d) Synthesis of (3S)-5-oxopyrrolidine-3-carboxamide (Compound (XIII))

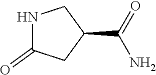

To a 500 L jacket reactor was charged with (3S)-5-oxo-1-(1-phenylvinyl)pyrrolidine-3-carboxamide (38.7 kg, 168 mol), methanol (108 kg) and hydrochloric acid (1.66 kg, 16.4 mol) at room temperature under N₂ atmosphere. The mixture was agitated using a magnetic stirrer at 20˜25° C. for 4 hours. Then the mixture was cooled to 0˜5° C., and agitated at that temperature for 3 hours. The solid was filtered and washed with methanol (40 kg) to give the desired product as off white solid, which was directly used for the preparation of Compound IV, giving 17 kg of (3S)-5-oxopyrrolidine-3-carboxamide. The purity was 99%, the chiral purity was 100%, the yield was 80%, and the MS m/e=129.1 [M+H]⁺. ¹H-NMR (400 MHz, DMSO-d⁶) δ 3.83; (dd, 1H, J=8.5, 13.8 Hz), 3.40; (dd, 1H, J=8.5, 13.8 Hz), 3.03; (m, 1H), 2.52; (dd, 1H, J=9.4, 18.8 Hz), 2.13; (dd, 1H, J=9.3, 18.8 Hz).

### Step e) Synthesis of (3S)-5-oxopyrrolidine-3-carbonitrile (Compound (IV))

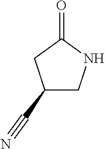

To a 200 L jacket reactor was charged with (3S)-5-oxopyrrolidine-3-carboxamide (17 kg, 132.7 mol) and MTBE (63 kg) at room temperature under N₂ atmosphere. The mixture was cooled to −5˜0° C. using Huber chiller. To the stirred mixture was charged with TFAA (70 kg, 333 mol) at −5˜0° C. The mixture was agitated using a magnetic stirrer at 20˜25° C. for another 4 hours. Then the mixture was cooled to −5˜0° C. again. The solid was filtered and washed with MTBE (30 kg) to give the desired product as off white solid, which was directly used for the preparation of compound (V), giving 11.7 kg of (3S)-5-oxopyrrolidine-3-carbonitrile. The purity was 99.3%, the chiral purity was 100%, the yield was 81%. MS m/e=110.1 [M+H]⁺. ¹H-NMR (400 MHz, DMSO-d⁶) δ=3.71; (dd, J=5.6, 9.2 Hz, 1H), 3.44; (dd, J=7.5, 9.2 Hz, 1H), 3.32; (m, 1H), 2.97; (dd, J=8.5, 18.2 Hz, 1H), 2.88; (dd, J=8.4, 18.2 Hz, 1H).

### Example 3

### tert-Butyl (6S)-6-methyl-3-[(4S)-4-cyano-2-oxo-pyrrolidin-1-yl]-6,7-dihydro-4H-pyrazolo[1,5-a]pyrazine-5-carboxylate (Compound (V))

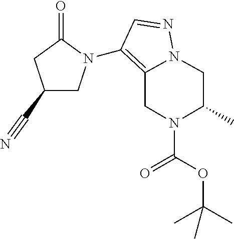 [SMILES: C[C@H]1Cn2ncc(N3C[C@@H](C#N)CC3=O)c2CN1C(=O)OC(C)(C)C]

To a 100 L jacket reactor was charged with THF (36 L), K₂CO₃ (5.14 kg), tert-butyl (6S)-3-iodo-6-methyl-6,7-dihydro-4H-pyrazolo[1,5-a]pyrazine-5- carboxylate (4.5 kg, 12.4 mol), (3S)-5-oxopyrrolidine-3-carbonitrile (2.04 kg, 18.5 mol), CuI (236 g, 1.24 mol) and N,N′-Dimethyl-1,2-ethanediamine (218 g, 2.5 mol) at room temperature under N₂ atmosphere. The reaction mixture was heated to 75° C. and the agitation was maintained for 1 hour. To the mixture was charged with another portion of N,N′-Dimethyl-1,2-ethanediamine (872 g, 10 mol) dropwise under refluxing. After being stirred under reflux for another 20 hours, the reaction mixture was slowly cooled to room temperature, and water (10 L) and IPAc (15 L) were added. After another 20 minutes, two layers were separated, and the organic layer was washed with 1N citric acid solution (6 L), 8% NaHCO₃ (8 L) solution and 10% brine (15 L). The organic layer was filtered through a Na₂SO₄ pad, and concentrated under reduced pressure to give the desired product as a light yellow solid, which was directly used for the preparation of compound (VI), giving 2.87 kg of tert-butyl (6S)-6-methyl-3-[(4S)-4-cyano-2-oxo-pyrrolidin-1-yl]-6,7-dihydro-4H-pyrazolo[1,5-a]pyrazine-5-carboxylate. The purity was 99%, the yield was 67%, and the MS m/e=345.2 [M+H]⁺. ¹H-NMR (400 MHz, DMSO-d⁶) δ=7.33; (s, 1H), 4.71; (d, J=16.8 Hz, 1H), 4.62; (m, 1H), 4.34; (dd, J=12.1, 12.5 Hz, 1H), 4.05; (dd, J=5.6, 9.2 Hz, 1H), 3.96; (dd, J=6.8, 12.1 Hz, 1H), 3.86; (d, J=16.8 Hz, 1H), 3.77; (dd, J=7.5, 9.2 Hz, 1H), 3.44; (m, 1H), 3.22; (dd, J=8.5, 18.2 Hz, 1H), 3.13 (dd, J=8.4, 18.2 Hz, 1H), 1.52; (d, J=6.6 Hz, 3H), 1.41; (s, 9H).

### Example 4

### (3S)-5-oxo-1-[(6S)-6-methyl-4,5,6,7-tetrahydropyrazolo[1,5-a]pyrazin-3-yl]pyrrolidine-3carbonitrile (Compound (VI))

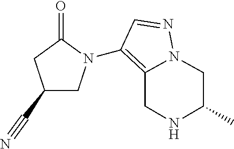 [SMILES: C[C@H]1Cn2ncc(N3C[C@@H](C#N)CC3=O)c2CN1]

To a 1L flask was charged with (6S)-6-methyl-3-[(4S)-4-cyano-2-oxo-pyrrolidin-1-yl]-6,7-dihydro-4H-pyrazolo[1,5-a]pyrazine-5-carboxylate (124 g, 359 mmol), HOAc (32.3 g, 539 mmol) and deionized water (620 mL) at room temperature. was After being stirred at 95° C. for 20 hours, the reaction mixture was cooled to room temperature and the water was azetroped with Toluene (300 mL×3). The resulting residue was triturated in MTBE (150 mL)/IPAc (150 mL) to give the desired product as a white solid, which was directly used for the preparation of compound (I), giving 1.6 kg of (3S)-5-oxo-1-[(6S)-6-methyl-4,5,6,7-tetrahydropyrazolo[1,5-a]pyrazin-3-yl]pyrrolidine-3-carbonitrile. The purity was 99.3%, the yield was 95%. MS m/e=245.1 [M+H]⁺. ¹H-NMR (400 MHz, DMSO-d⁶) δ=7.32; (s, 1H), 4.19; (dd, J=11.1, 16.4 Hz, 1H), 4.06; (dd, J=4.3, 16.4 Hz, 1H), 4.00; (dd, J=5.6, 9.2 Hz, 1H), 3.96; (dd, J=3.5, 12.4 Hz, 1H), 3.72; (dd, J=7.5, 9.2 Hz, 1H), 3.63; (dd, J=10.5, 12.4 Hz, 1H), 3.28; (m, 1H), 3.28; (m, 1H), 3.17; (dd, J=8.5, 18.2 Hz, 1H), 3.08; (dd, J=8.4, 18.2 Hz, 1H), 1.02; (d, J=6.4 Hz, 3H).

### Example 5

### Phenyl N-(3,4,5-trifluorophenyl)carbamate (Compound (VII))

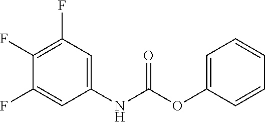 [SMILES: O=C(Nc1cc(F)c(F)c(F)c1)Oc1ccccc1]

To a 20 L jacket reactor was charged with 3,4,5-trifluoroaniline (1 kg, 3.8 mol) and THF (10 L) at room temperature under N₂ atmosphere. The mixture was cooled to −5˜0° C. To the stirred mixture was charged with an aqueous solution of NaHCO₃ (856 g, 10.2 mol) in water (5 L) and phenyl carbonochloridate (1.2 kg, 7.48 mol) at −5˜0° C. After the addition, the reaction mixture was stirred at 0˜10° C. for another 1 hour, then EtOAc (5 L) and water (2 L) were added.

Two layers were separated, and the organic layer was washed with sat. NaCl (6 L), filtered through a Na₂SO₄ pad, and concentrated under reduced pressure to give the desired product as white solid, which was directly used for the preparation of compound (I), giving 0.9 kg of phenyl N-(3,4,5-trifluorophenyl)carbamate. The purity was 99%, the yield was 92%, and the MS m/e=267.1 [M+H]⁺. ¹H-NMR (400 MHz, CDCl₃) δ=7.43; (dd, J=7.5, 8.1 Hz, 1H), 7.43; (dd, J=7.5, 8.1 Hz, 1H), 7.28; (dd, J=7.5, 12.3 Hz, 1H), 7.28; (dd, J=7.5, 12.3 Hz, 1H), 7.20; (t, J=7.5 Hz, 1H), 7.19; (dd, J=2.7, 8.1 Hz, 1H), 7.19; (dd, J=2.7, 8.1 Hz, 1H).

### Example 6

### (6S)-3-[(4S)-4-cyano-2-oxo-pyrrolidin-1-yl]-6-methyl-N-(3,4,5-trifluorophenyl)-6,7-dihydro-4H-pyrazolo[1,5-a]pyrazine-5-carboxamide (Compound (I))

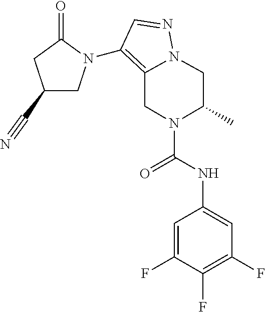 [SMILES: C[C@H]1Cn2ncc(N3C[C@@H](C#N)CC3=O)c2CN1C(=O)Nc1cc(F)c(F)c(F)c1]

To a 50 L jacket reactor was charged with (3S)-5-oxo-1-[(6S)-6-methyl-4,5,6,7-tetrahydropyrazolo[1,5-a]pyrazin-3-yl]pyrrolidine-3-carbonitrile (2.2kg, 9 mol), phenyl N-(3,4,5-trifluorophenyl)carbamate (2.4 kg, 9 mol), IPAc (19.1 kg), acetone (3.3 kg) and DIPEA (1.39 kg) under N₂ atmosphere. The mixture was agitated using a magnetic stirrer at 45˜50° C. for 20 hours.

Then the mixture was cooled to room temperature slowly. The white solid was filtered and washed with IPAc:MTBE (V/V=2/1, 6.7 kg). The wet cake was dried in a vacuum oven (IT=50 ° C.) to give the desired product as a white solid, giving 3.5 kg of (6S)-3-[(4S)-4-cyano-2-oxo-pyrrolidin-1-yl]-6-methyl-N-(3,4,5-trifluorophenyl)-6,7-dihydro- 4H-pyrazolo[1,5-a]pyrazine-5-carboxamide. The purity was 98.5%, the chiral purity was 100%, the yield was 93%. MS m/e=418.1 [M+H]⁺. ¹H-NMR (400 MHz, DMSO-d⁶) δ=7.38; (s, 1H), 7.16; (dd, J=7.4, 13.4 Hz, 1H), 7.16; (dd, J=7.4, 13.4 Hz, 1H), 4.43; (m, 1H), 4.29; (d, J=16.8 Hz, 1H), 4.13; (dd, J=12.1, 12.5 Hz, 1H), 3.99; (dd, J=5.6, 9.2 Hz, 1H), 3.84; (d, J=16.8 Hz, 1H), 3.80; (dd, J=6.8, 12.1 Hz, 1H), 3.71; (dd, J=7.5, 9.2 Hz, 1H), 3.44; (m, 1H), 3.22; (dd, J=8.5, 18.2 Hz, 1H), 3.13; (dd, J=8.4, 18.2 Hz, 1H), 1.31; (d, J=6.6 Hz, 3H).

1. Process for the preparation of a compound (I), 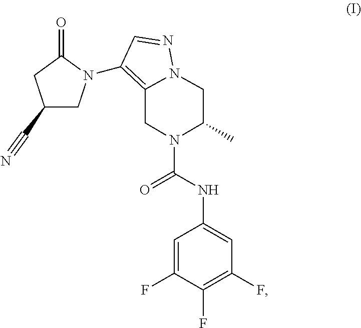 [SMILES: C[C@H]1Cn2ncc(N3C[C@@H](C#N)CC3=O)c2CN1C(=O)Nc1cc(F)c(F)c(F)c1]
or pharmaceutically acceptable salt thereof;
comprising the following steps:
step 1) the formation of compound (III), 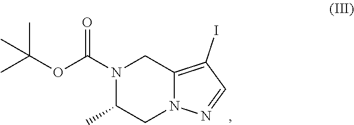 [SMILES: C[C@H]1Cn2ncc(I)c2CN1C(=O)OC(C)(C)C]
via iodization reaction of compound (II), 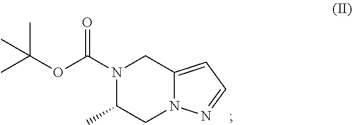 [SMILES: C[C@H]1Cn2nccc2CN1C(=O)OC(C)(C)C]
step 2) the formation of compound (V), 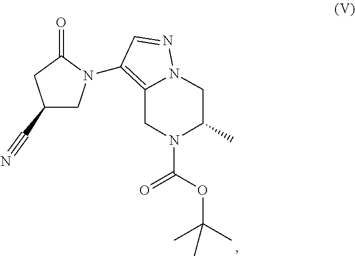 [SMILES: C[C@H]1Cn2ncc(N3C[C@@H](C#N)CC3=O)c2CN1C(=O)OC(C)(C)C]
via Ullmann reaction between compound (III) and compound (IV), 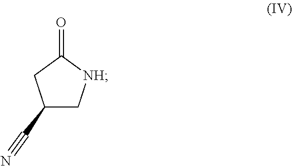
step 3) the formation of compound (VI), 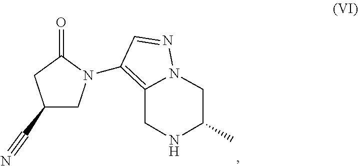 [SMILES: C[C@H]1Cn2ncc(N3C[C@@H](C#N)CC3=O)c2CN1]
via de-protection of compound (V);
step 4) the formation of compound (VII), 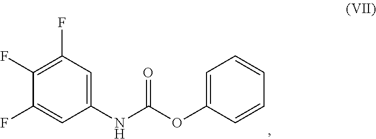 [SMILES: O=C(Nc1cc(F)c(F)c(F)c1)Oc1ccccc1]
via the reaction between 3,4,5-trifluoroaniline and phenyl carbonochloridate;
step 5) the formation of compound (I), 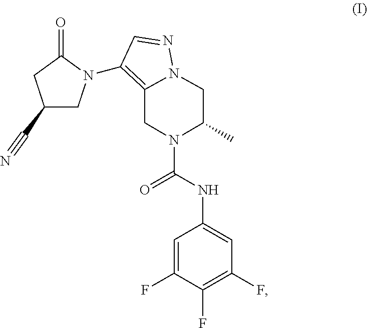 [SMILES: C[C@H]1Cn2ncc(N3C[C@@H](C#N)CC3=O)c2CN1C(=O)Nc1cc(F)c(F)c(F)c1]
via the substitution reaction between compound (VI) and compound (VII).

2. A process according to claim 1, characterized in that the formation of compound (III) in step 1) is performed in the presence of an iodization reagent, wherein the iodization reagent is selected from NIS and I₂; particularly the iodization reagent is NIS.

3. A process according to claim 1 or 2, characterized in that the excessive I₂ formed during the reaction due to the use of iodization reagent in step 1) is removed by Na₂SO₃, wherein the amount of Na₂SO₃ is 0.4-0.8 eq., particularly 0.55-0.6 eq.

4. A process according to any one of claims 1 to 3, characterized in that the formation of compound (V) in step 2) is performed in the presence of a base, a catalyst and a ligand in an organic solvent; wherein the base is selected from K₂CO₃, K₃PO₄ and Cs₂CO₃, particularly the base is K₂CO₃; wherein the catalyst is CuI and the amount of catalyst is 0.05-0.5eq, particularly 0.1 eq.; wherein the ligand is DMEDA and the amount of ligand is 0.2-2.0 eq, particularly 1.0 eq.; wherein the organic solvent is selected from 1,4-Dioxane, ACN, Toluene, THF and MeTHF, particularly the organic solvent is THF.

5. A process according to any one of claims 1 to 4, characterized in that the formation of compound (VI) in step 3) is performed in the presence of an acid, wherein the acid is selected from H₃PO₄, NH₄Cl, TFA and acetic acid, particularly the acid is acetic acid; wherein the amount of acid is 1˜3 eq., particularly 1.5˜2 eq.

6. A process according to any one of claims 1 to 5, characterized in that the formation of compound (VII) in step 4) is performed in the presence of a base, wherein the base is selected from K₂CO₃, KHCO₃, NaHCO₃ and Na₂CO₃, particularly the base is NaHCO₃.

7. A process according to claim 6, characterized in that the formation of compound (VII) in step 4) is performed at −10° C.˜10° C., particularly at −5˜0° C.

8. A process according to any one of claims 1 to 7, characterized in that the formation of compound (I) in step 5) is performed in the presence of a base, wherein the base is selected from K₂CO₃, K₃PO₄, DIPEA and TEA, particularly the base is DIPEA.

9. Process for the preparation of a compound (IV), 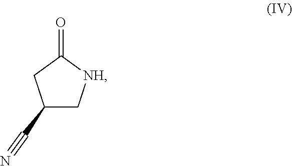
or pharmaceutically acceptable salt thereof;
comprising the following steps:
step a) the formation of compound (X), 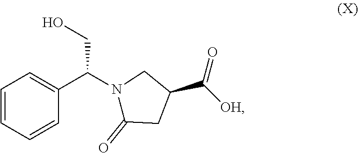 [SMILES: O=C(O)[C@H]1CC(=O)N([C@@H](CO)c2ccccc2)C1]
via cyclization reaction between compound (VIII), 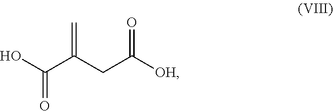
and compound (IX), 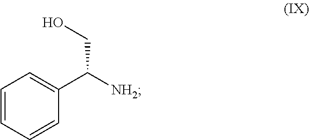
step b) the formation of compound (XI), 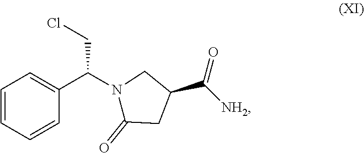 [SMILES: NC(=O)[C@H]1CC(=O)N([C@@H](CCl)c2ccccc2)C1]
via substitution of compound (X);
step c) the formation of compound (XII), 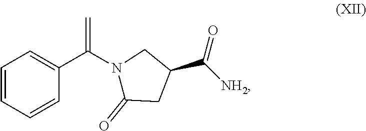 [SMILES: C=C(c1ccccc1)N1C[C@@H](C(N)=O)CC1=O]
via elimination reaction of compound (XI);
step d) the formation of compound (XIII), 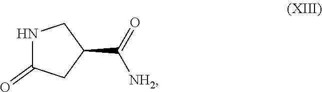
by deprotection of compound (XII);
step e) the formation of compound (IV), 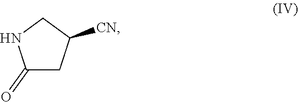
via dehydration of compound (XIII)

10. A process according to claim 9, characterized in that the formation of compound (X) in step a) is performed via cyclization reaction in an organic solvent, wherein the solvent is selected from THF, ACN and MeTHF, particularly the organic solvent is ACN; wherein the cyclization reaction is performed at 80˜140° C., particularly at 100˜110° C.

11. A process according to claim 9 or 10, characterized in that the formation of compound (XI) in step b) is performed in the presence of chlorination reagent, wherein the chlorination reagent is selected from acetyl chloride, thionyl chloride and oxalyl chloride, particularly the reagent is thionyl chloride; wherein the amount of the chlorination reagent is 1˜5 eq., particularly 3 eq.

12. A process according to any of claims 9 to 11, characterized in that the formation of compound (XII) in step c) is performed in the presence of a base, wherein the base is selected from K₃PO₄, TEA, DBU and DIPEA, particularly the base is DBU; wherein the amount of base is 1˜2 eq., particularly 1.5 eq.

13. A process according to any of claims 9 to 12, characterized in that the formation of compound (XIII) in step d) is performed in the presence of an acid, wherein the acid is selected from H₂SO₄, H₃PO₄, HCl and TFA, particularly the acid is HCl; wherein the amount of acid is 0.05˜0.5eq., particularly 0.1 eq.

14. A process according to any of claims 9 to 13, characterized in that the formation of compound (IV) in step e) is performed in the presence of a dehydration reagent, wherein the dehydration reagent is selected from P₂O₅, TFAA and acetic anhydride, particularly the dehydration reagent is TFAA; wherein the amount of dehydration reagent is 2.5 eq.
